(19) United States
(12) Patent Application Publication
(10) Pub. No.: US 2002/0110931 A1
(43) Pub. Date: Aug. 15, 2002

(54) Coded testing apparatus

(75) Inventors: Richard A. Quattrocchi, Barrington, IL (US); Michael Wandell, Mercer Island, WA (US)

Correspondence Address: LEYDIG VOIT & MAYER, LTD, TWO PRUDENTIAL PLAZA, SUITE 4900, 180 NORTH STETSON AVENUE, CHICAGO, IL, 60601-6780, US

(73) Assignee: Home Access Health Corporation, Hoffman Estates, IL (US)

(21) Appl. No.: 10/108,074

(22) Filed: Mar. 27, 2002

Related U.S. Application Data
(62) Division of application No. 09/782,401, filed on Feb. 13, 2001.

(51) Int. Cl. G01N 33/543; G01N 33/00

(52) U.S. Cl. 436/518; 436/183

(57) ABSTRACT

Disclosed is a coded testing apparatus. In accordance with the disclosed invention, a user may perform a medical test which yields an encoded test result. The user then communicates the encoded test result to a facility, whereupon the facility provides the result to the user. The invention allows for many of the advantages of “instant” medical testing, such as convenience and anonymity, but avoids substantial drawbacks associated with known “instant” tests, such as the inability to request demographic information, the inability to provide appropriate medical oversight, and the inability to provide adequate medical counseling.

### TECHNICAL FIELD OF THE INVENTION

The invention is in the field of testing, and, in particular, the preferred embodiments of the invention are in the field of medical testing. Generally, the invention pertains to a coded testing apparatus that is particularly suitable for use in connection with a telemedical testing service.

### BACKGROUND OF THE INVENTION

Medical technology has advanced to the point where many medical conditions and diseases now can be accurately diagnosed using “instant” medical tests. Using a suitable testing apparatus, a bodily specimen, such as blood, serum, plasma, urine, semen, saliva, buccal or oral mucosal cells, or sweat is collected and introduced onto or into a specimen tester. The specimen tester includes chemicals which react with chemical components present in the bodily specimen to thereby provide a visual indicator from which the test results can be determined. One familiar application of such technology is a home pregnancy test. Typically, such test provides an indicator display that includes plural indicators, most typically a positive control indicator that becomes colored upon reaction with components expected to be present in all bodily fluids to thus signify that the test has been performed accurately, and a positive indicator that changes color depending on the presence of absence of components in the bodily fluid. In connection with one commercially available home pregnancy test, the positive and positive control indicators are combined such that the testing apparatus will display a “minus” sign (which consists of the control indicator) to indicate non-pregnancy and a “plus” sign (which consists of the positive control indicator and two adjacent abutting positive indicators) to indicate pregnancy.

Beyond such pregnancy tests, the state of medical technology has now advanced to the point that instant tests are now available for a variety of complex conditions, such as viral infection with HIV (Human Immunodeficiency Virus), infection with the hepatitis virus, as well as other conditions that may indicate disorders such as diabetes. As the state of medical technology continues to advance, it is believed that other instant tests will become available. For example, it is envisioned that graded tests, i.e., tests that provide a grade or “score” such as a blood cholesterol test, may be developed in addition to new types of tests designed to indicate the presence or absence of a specific medical condition. Instant graded tests for determining the level of alcohol in the bloodstream already have been developed and are in use by law enforcement officials.

Known instant tests offer a number of advantages, including convenience for the person taking the test, the lack of required participation by trained medical personnel, and the potential for the test-taker to remain anonymous. However, as the state of medical technology has continued to develop, a number of concerns have arisen over the use of such tests. Presently, there exist serious reservations about placing powerful diagnostic tests in the hands of laypersons, who may not appreciate the intricacies of such tests or the limitations of the test technology. Moreover, significant questions concerning such tests need to be addressed, such as questions concerning access to professional medical oversight, the proper interpretation of the results of such tests, the gathering of statistics and demographic information concerning test takers and test results (information which is of importance to governmental health bodies and other health organizations), and the provision of adequate counseling to persons who have taken such tests, particularly in the case of a positive diagnosis of a disorder such as a viral infection or genetic condition. It is believed that such concerns have led to a general reluctance to widely disseminate “instant” testing technology to the general public.

It is a general object of the invention to provide a testing apparatus that is suitable for use by laypersons but that addresses the concerns that have arisen in connection with home use of self-performed diagnostics. In another embodiment, it is a general object of the invention to provide a method for processing incoming test result inquiries from a plurality of users of such apparatus.

### THE INVENTION

The present invention is premised upon the concept that “instant” tests can be conduced by laypersons using a testing apparatus that provides for encoded test results that the layperson ordinarily cannot interpret directly. After testing a specimen of blood, urine or the like with the testing apparatus of the invention, the user receives an encoded result, and must contact an appropriate facility to provide the encoded result to the facility in order to obtain the results of the test. Most preferably, the testing apparatus is identified with a code, which may be either a unique code, a lot number of manufacture of the device, or both. In this preferred embodiment, the facility, based on the code and the encoded test result provided by the user, can determine the result of the test, and can provide the test result to the user. In this respect, appropriate safeguards can be taken to ensure that demographic information concerning the user is collected, that appropriate medical oversight and counseling are available, and that the test results are interpreted properly. Thus, many of the advantages of “instant” or “point-of-care” testing can be realized, including the option for the user to remain anonymous, but many of the concerns that exist with regard to such tests can be mitigated.

In accordance with one aspect of the invention, a testing apparatus is provided. The testing apparatus comprises a specimen tester that has a non-specific result display and a code for use in obtaining test result information from an external source from the nonspecific test result display. Preferably, the specimen tester includes plural indicators, including at least one positive indicator and at least one control indicator; a first non-specific identifier associated with the control indicator; and a second non-specific identifier associated with the positive indicator. The testing apparatus may be provided in the form of a kit, which may include a specimen collector, a developer, and other appropriate components. The invention further encompasses a method for manufacturing a testing apparatus.

In accordance with another aspect of the invention, a method for processing a plurality of in-coming inquiries from users of a testing apparatus desired above is provided. Generally, the method comprises receiving the code number and the test result indicator information from the user, determining the test result from this information, and providing the test result to the user. The method of this aspect of the invention can be practiced by a results providing facility. The invention thus is particularly applicable in the “telemedicine” arena, in which medical test result information or other medical information is provided by telephone or other electronic communication, such as e-mail, web-site posting, or the like. Three pioneering patents in the field of telemedicine are U.S. Pat. Nos. 6,014,438, 6,016,345, 5,978,466, assigned to Home Access Health Corporation of Hoffman Estates, Illinois. The method of the present invention may be used in conjunction with a method for routing inquiries disclosed in these patents.

### DESCRIPTION OF THE DRAWINGS

FIG. 1 is a perspective view of one embodiment of the testing apparatus of the invention.

FIG. 2 is a front elevation of the testing apparatus shown in FIG. 1.

FIG. 3 is a rear elevation of the testing apparatus shown in FIG. 1.

FIG. 4 is an idealized perspective view of a kit in accordance with another embodiment of the invention.

FIG. 5 is a table of indicator results possible for a testing apparatus that includes one positive indicator and one control indicator.

FIGS. 6A and 6B are tables representing the two alternative keys to the encoding scheme for the test results tabulated in FIG. 5.

FIG. 7 is a table of indicator results possible for a testing apparatus that includes one positive indicator and two control indicators.

FIGS. 8A, 8B, and 8C are tables representing three alternative keys to the encoding scheme for the test results tabulated in FIG. 7.

FIG. 9 is a table of test results possible for a testing apparatus that includes one positive indicator and three control indicators.

FIG. 10 is a flow chart that represents the steps in a method for processing in-coming inquiries.

### DESCRIPTION OF PREFERRED EMBODIMENTS

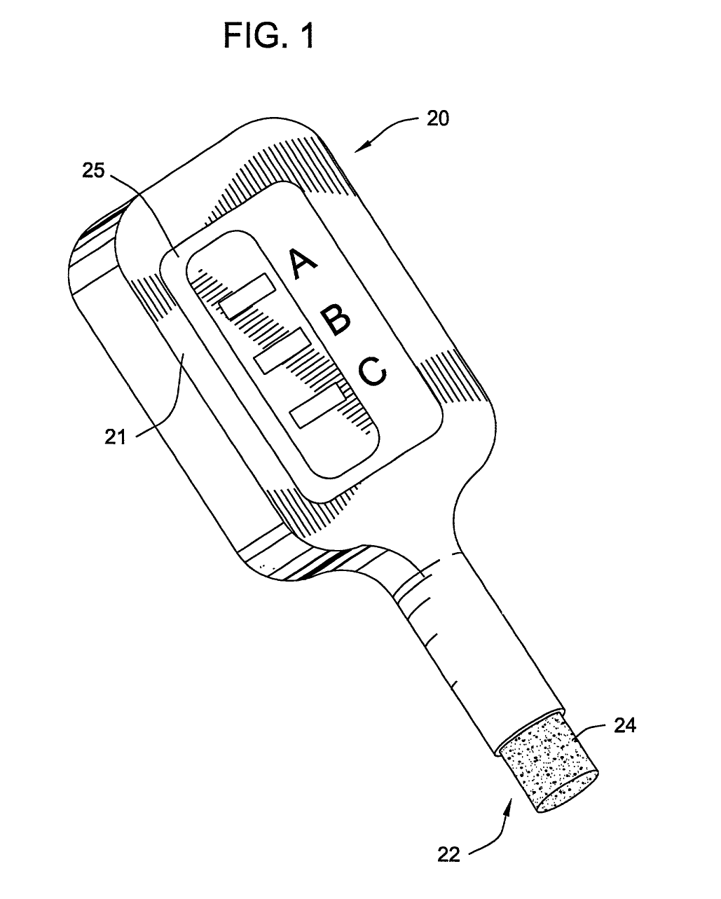

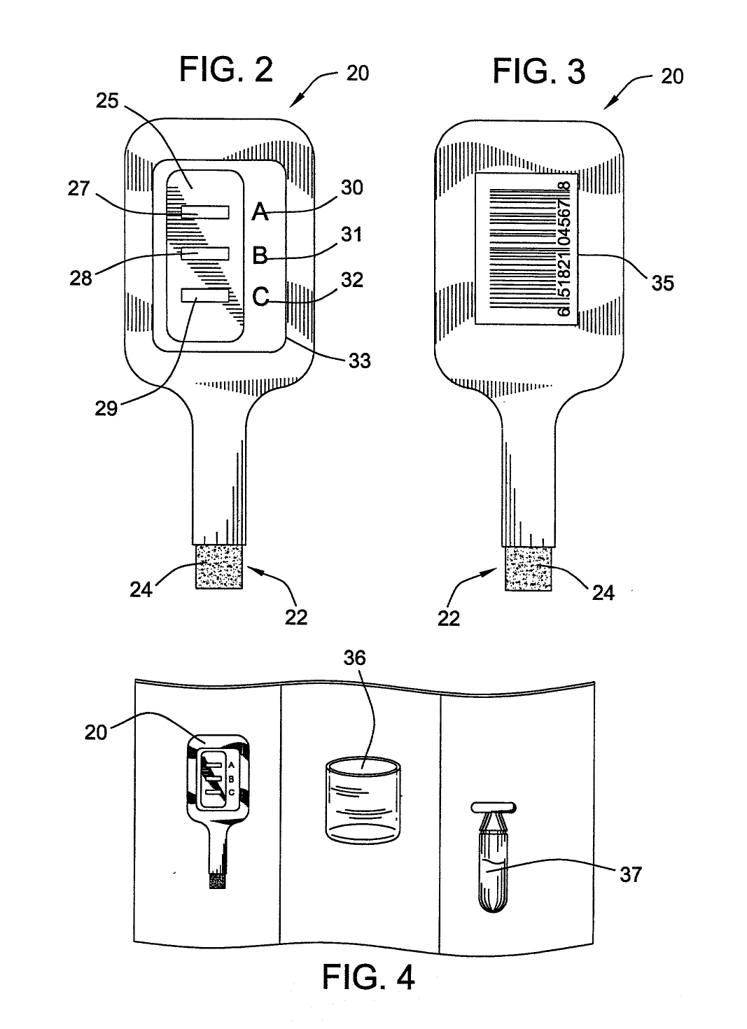

As shown in FIGS. 1-3, the testing apparatus 20 generally comprises a housing 21 and a specimen tester 22, the specimen tester generally including a portion 24 for receiving a specimen of blood, urine, or other fluid or other specimen, and a display portion 25. For instance, when the apparatus is used in connection with a hepatitis test, specific recombinant proteins and peptides for Hepatitis C (manufactured by Chiron Corporation, for example) are applied to the paper that will react in the presence of antibodies and fluid to create a color change.

The illustrated apparatus includes three indicator lines, which include a positive indicator 27, which changes color if the test result is positive; a positive control indicator 28, which changes color if the test has been performed correctly; and a negative control indicator 29, which alternatively may be an indicator that does not change color, or an indicator that changes color only if the test has been done incorrectly (for instance, if the user has permitted excess specimen to be introduced onto the specimen tester). It is contemplated with respect to the positive indicator that for certain types of tests such indicator might become colored or otherwise altered from its original configuration in the event that a certain component is not present in the specimen being tested. Thus, although the test might be deemed “negative,” for example, to thus indicate lack of infection with a specific pathogen, the indicator that becomes colored in such case to indicate the absence of such pathogen in the specimen is still referred to herein as being as the “positive” indicator. In other embodiments, a control indicator may be designed to change to a first color if the test is properly performed and to a second color if the test is improperly performed. Also, it is contemplated that an indicator may be designed to change to one of plural colors depending on the grade of the sample.

Although three indicators are shown in the illustrated embodiment, it is contemplated that two indicators, four indicators, or more than four indicators could be present, but generally at least one positive indicator and at least one second indicator should be present. The positive indicator 27 and control indicators 28, 29 have been identified with specifically herein, but the user of the apparatus ordinarily would not be informed which the indicators is positive and which are the control indicators. Instead, the apparatus includes three nonspecific identifiers 30, 31, 32, which in the illustrated embodiment are shown as letters A-C. The identifiers 30, 31, 32 are associated respectively with the indicators 27, 28, 29, by which is contemplated that a user will perceive that each identifier identifies the respective indicator, but the identifiers are non-specific, i.e. they do not provide information as to the significance of a change in indicators. The results of the test thus will be encoded, and the user ordinarily will not be able to ascertain the test result. The indicators may be provided in the form of a film sticker 33. In this respect, it is possible for numerous identical specimen testers to be manufactured, and these provided with various different stickers that contain different identifiers, or that contain the same identifiers in a different order. In either such case, a user who was not aware that his or her testing apparatus included a specimen tester identical to those found in other testing apparati ordinarily would not know how to interpret the results provided by the various indicators.

The apparatus further is provided with a code, which, in the illustrated embodiment, is provided in the form of a bar code sticker 35 that contains a numeric code. The code is reported to a result providing facility in combination with the encoded test results on the display portion 25 to determine the actual result of the test. In this respect, the code number can uniquely identify the testing apparatus, in which case the testing apparatus and the encoding key information (such as the number, type, and/or order of indicators) are associated with the code number in a database that is not accessible to the user. Alternatively, the code number can identify the testing apparatus by lot, in which case the specific encoding key information need only be paired with lot information. In a further alternative embodiment, or in addition thereto, the code number itself could intrinsically contain encoding key information for the testing apparatus. In such case, the result providing facility would be provided with an algorithm for deciphering the code number.

FIG. 4 illustrates a kit that may be provided to a user in connection with the present invention. The kit includes in a package the testing apparatus 20 shown in FIG. 1; a specimen collector 36, which, in the illustrated embodiment, comprises a cup for urine collection, and a sample developer 37, which may be required for certain types of tests (for instance, an HIV test presently requires development of a urine sample with an added reagent). In some embodiments, a specimen collector may be omitted; in other embodiments, a specimen developer may be omitted. The kit may further include a quality control device, such as an image, that the user may be asked to identify upon contacting the results providing facility. For instance in the case of a test for strep throat, the user may be asked to identify an insert resembling the patient's throat. If the use is unable to identify the image, such is an indication that the user is unable to conduct the test properly. It is envisioned that the kit may further include other components, such as instructions, a desiccant for retarding moisture spoilage, or other components. In another embodiment of the invention, the kit comprises the testing apparatus and instructions for use, the instructions including directions for contacting a results providing facility. Preferably, the instructions do not including directions for allowing the user to interpret the results directly. The kit may further include a specimen collector, a specimen developer, and/or other suitable components.

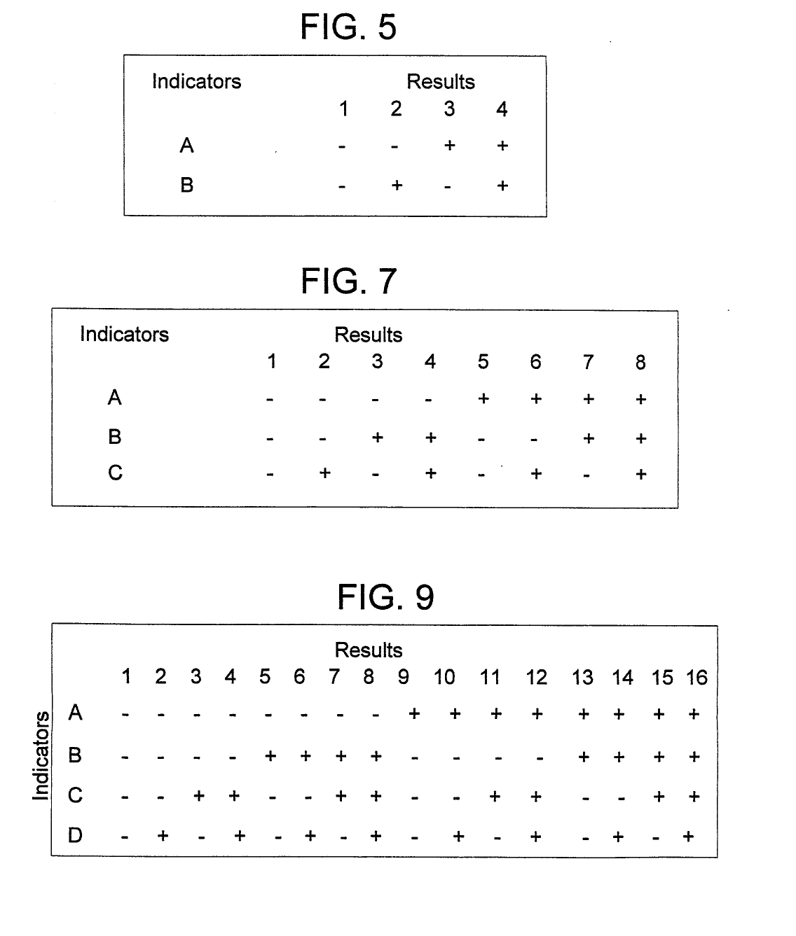

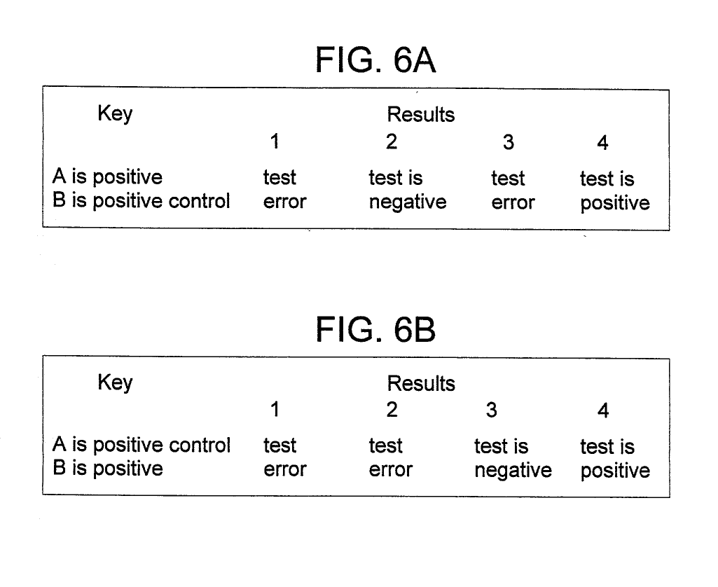

FIG. 5 tabulates all of the conceivably possible test results for a testing apparatus that includes two indicators in which each indicator either turns colored (signified by a plus (+) sign) or remains uncolored (signified by a minus (−) sign). The test results set forth in the table may be interpreted with respect to the encoding key information tables set forth as FIGS. 6A and 6B, which tester may be stored in a database at a results providing facility. As set forth, for instance, in FIG. 6A, the testing apparatus might include two indicators wherein “A” is the positive indicator and wherein “B” is the positive control indicator. In this case, result 2 would correspond to a negative test result, and result 4 to a positive test result. Results 1 and 3 (wherein the positive control indicator is negative) should not be observed for a properly performed test or properly interpreted test results and would signify a test error. As shown in FIG. 6B the indicators would be otherwise construed if indicator “A” is the positive control indicator and if indicator “B” is the positive indicator.

Generally, it is preferred that the testing apparatus include at least one positive control indicator in addition to the positive indicator, so that the accuracy of the testing method can be validated. Many laypersons are sophisticated concerning testing methods and will be aware of the likelihood that testing apparatus will include at least one positive control indicator and one positive indicator. Accordingly, such users may be able to interpret the test result without needing to contact a results providing facility for interpretation of the encoded test results. This presents a potential difficulty in maintaining the intended testing protocol, but this difficulty can be mitigated if the testing apparatus includes additional indicators, the nature of which are not disclosed to the user. For instance, the testing apparatus may be provided with a third, fourth, fifth, etc. indicator, each of which may be a positive indicator, a positive control indicator, or a negative control indicator. FIGS. 7 and 9 respectively tabulate the possible encoded test results for a three- and four-indicator testing apparatus in which each indicator either turns colored (signified by a plus (+) sign) or remains uncolored (signified by a minus (−) sign). While it sometimes may be possible for a user to analyze the encoded test results, such is less likely and in some instances impossible without contacting the results providing facility. For instance, with respect to result 13 in FIG. 9, the user is not able to determine whether indicators A and B are a positive control and positive indicator (which would signify a positive test result) or whether both are positive control indicators (which, given that the positive indicator would be one of indicators C and D, would signify a negative result).

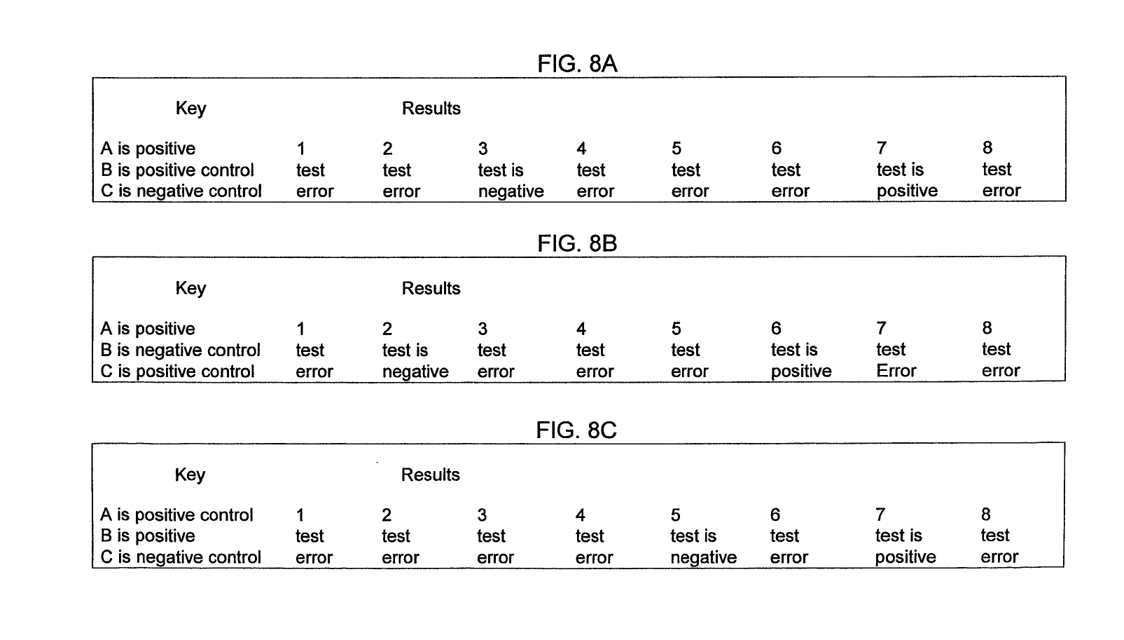

The results can be interpreted by a results providing facility with the aid of interpretive tables, such as those set forth for a three indicator apparatus at FIGS. 8A-C (other tabulations would be provided for other configurations, such as when indicator “A” is a negative control indicator). Tables of encoding key information that correspond to each of the permutations of indicators in the testing apparati that have been distributed may be stored in a database at the results providing facility. The database further may contain records that associate the encoding key information with code lots (for instance, a first lot of codes may be associated with the results tabulated in FIG. 8A, a second lot with the results tabulated in FIG. 8B, and a third lot with the results tabulated in FIG. 8C).

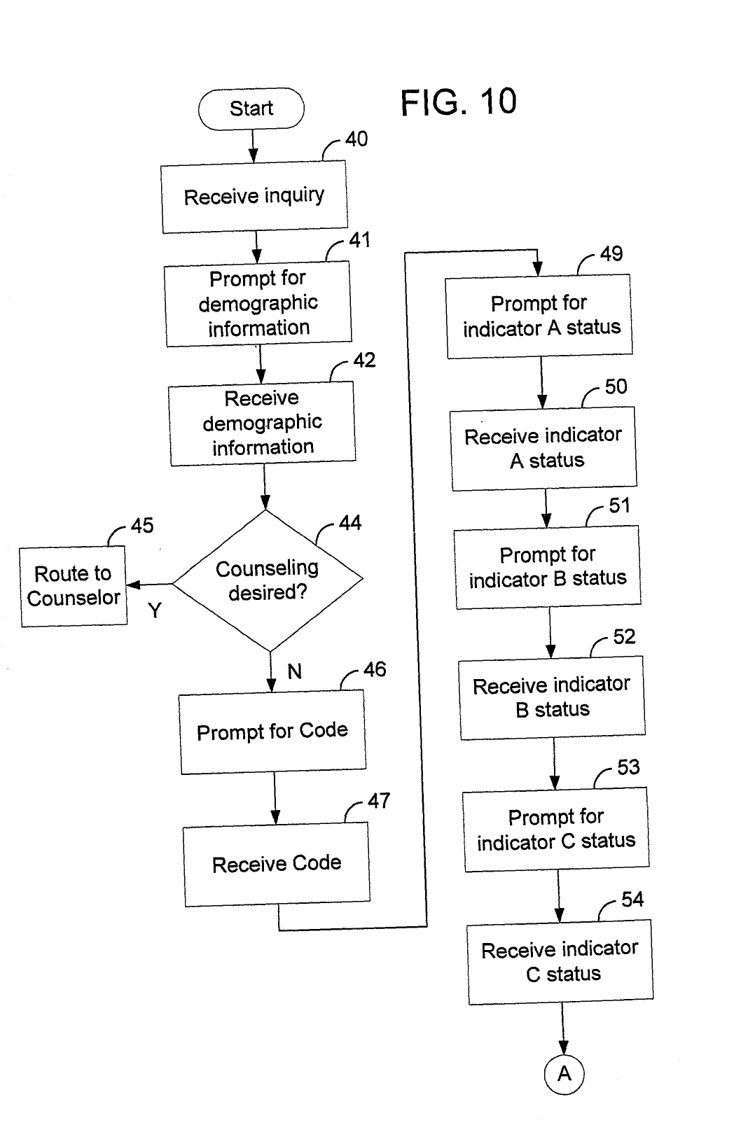

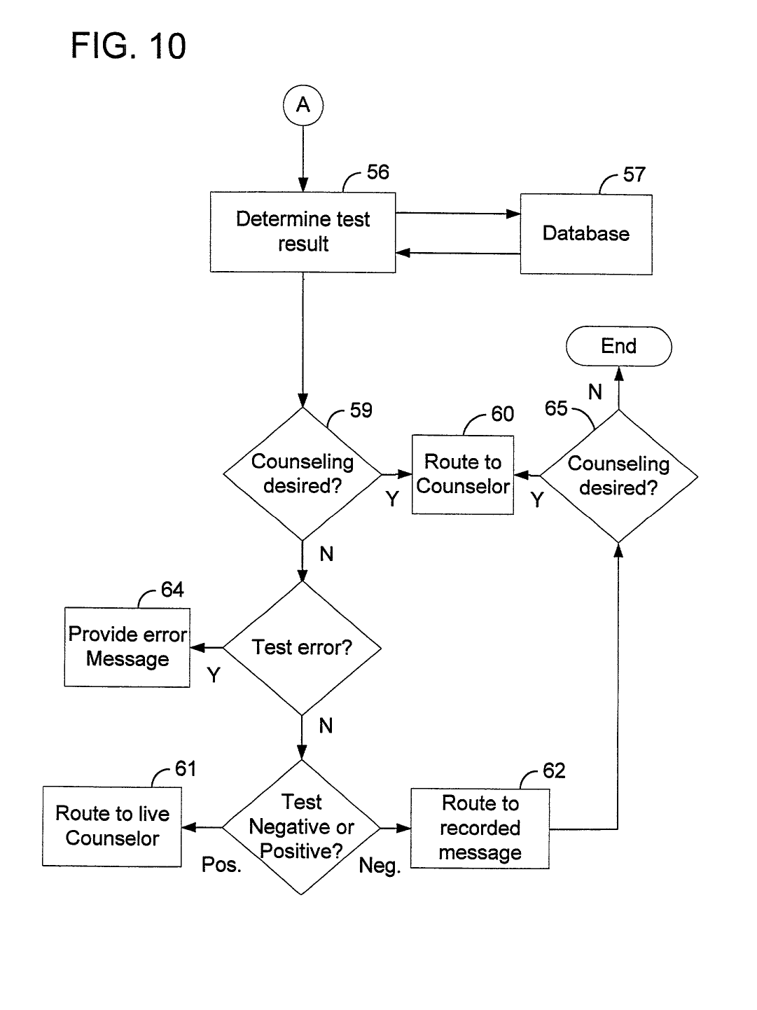

The invention further contemplates a method for processing in-coming inquiries from users of a test apparatus as described above. Most preferably, in accordance with the method of the invention, a testing kit is distributed to each of plural users. The identities of the users may be known and identified with the code on the testing kit, or the users may be anonymous, which includes not only the case where no information about the test-taker is known but also cases where some information is known, for instance, where testing kits are distributed anonymously to a plurality of known users. For example, the users may be employees of a company participating in a testing program. In accordance with one aspect of the invention, a method for processing a plurality of incoming inquiries from such users is provided. As shown in FIG. 10, at step 40 an in-coming inquiry is received. The inquiry may be received and processed by any suitable equipment, such as a computer system equipped with message handling equipment (for instance, automated touch-tone telephone handlers, interfaces for processing messages over a computer network, such as the World Wide Web, or other appropriate handling equipment). At step 41 the user is prompted for demographic information, such as race, age, gender, and so forth; this information is received at step 42. At appropriate steps in the process, the user may be prompted as to whether the user desires counseling; for example, at step 44 in the illustrated embodiment, the user is prompted as to whether counseling is desired. If counseling is desired, the user is routed to a counselor at step 45 (in the illustrated embodiment, the method is terminated once the inquiry has been routed to a counselor). Otherwise, control passes to step 46 when the user is prompted for his or her code, which is received at step 47. Subsequently, the user is prompted for the indicator result for each of the three indicators A, B, C; these occurring at steps 49, 51, and 53. After receiving the indicator information at steps 50, 52, and 54 respectively, the test result is determined at step 56. If the encoding key information is stored in a database, reference is made to the database 57; alternatively, where the code obtains intrinsic information concerning the indicators, the test result would be determined via an appropriate algorithm (step not shown). The user again is prompted as to whether counseling is desired at step 59. If counseling is desired, the call is routed to a counselor at step 60. Otherwise, the test result is provided to the user. In the case of a medical test, such as a test for hepatitis or HIV, the system may be operated such that the call is routed to a live counselor at step 61 if the test result is positive, or to a recorded message 62 if the test result is negative. The user may be routed to an error call handler at step 64 if the test is deemed to have been performed incorrectly. The user again may be prompted for counseling at step 65, with counseling provided (shown again at step 60) if requested. Further details concerning the routing of user inquires based on test results are set forth in U.S. Pat. Nos. 6,014,438, 6,016,345 and 6,016,345, identified hereinabove.

The foregoing steps may be performed in any suitable order, and steps may be added or omitted as deemed appropriate. For instance, the code number may be requested subsequent to the entry of indicator information; counseling steps may be added or omitted; and, more generally, the method may be otherwise performed in any manner deemed suitable. The operation paths shown in the Figures are exemplar and do not represent all possible options. The paths taken by the automated system are various and may be dynamic based on various combinations of the stored data parameters and user inputs.

Thus, it is seen that the foregoing general object has been satisfied. A testing apparatus that makes possible many of the advantages of “instant” testing but that avoids many of the drawbacks of such testing is provided. Moreover, it is contemplated that other embodiments of the invention are possible. The invention finds applicability to other fields of testing besides human medical testing, such as environmental, animal health, nutritional or other testing. The indicators may be provided in other forms, such as in the form of multicolored indicators that change colors depending on the presence of absence of a detected component of the specimen or based on the “grade” of the specimen. The code may be provided other than in the form of a number, such as an alphanumeric code. Communications to and from the results providing facility, such as communications by the user or counseling communications from the facility, may be provided by telephone; by electronic communication, such as electronic mail or electronic “chat”; or by other suitable means. The “display” can be not only a visual display, but also or alternatively an aural display or other type of display. More generally, while particular embodiments of the invention have been shown, it will be understood that the invention is not limited thereto since modifications may be made by those skilled in the art, particularly in light of the foregoing teachings. It is, therefore, contemplated by the appended claims to cover any such modifications as incorporate those features which constitute the essential features of these improvements within the true spirit and scope of the invention. All references cited herein are herby incorporated by reference in their entireties.

What is claimed is:

1. A testing apparatus comprising:
a specimen tester, said specimen tester including at least one control indicator and at least one positive indicator;
a first non-specific identifier associated with said control indicator;
a second non-specific identifier associated with said positive indicator; and
a code for obtaining test result information from an external source based on said first and second indicators.

2. A testing apparatus according to claim 1, said code identifying said testing apparatus from among a plurality of testing apparati.

3. A testing apparatus according to claim 2, said code identifying said testing apparatus by lot.

4. A testing apparatus according to claim 1, said code including intrinsic encoding key information for said first and second identifiers and said indicators.

5. A testing apparatus according to claim 1, wherein said control indicator is a positive control indicator.

6. A testing apparatus according to claim 1, further including a third indicator selected from a group consisting of a positive indicator, a positive control indicator, and a negative control indicator, the apparatus including a third non-specific identifier associated with said third indicator.

7. A testing apparatus according to claim 6, further including a fourth indicator, said fourth indicator being selected from the group consisting of a positive indicator, a positive control indicator and a negative control indicator, the apparatus including a fourth non-specific identifier associated with said fourth indicator.

8. A kit comprising:
a testing apparatus, said testing apparatus comprising a specimen tester, said specimen tester including at least one control indicator and at least one positive indicator;
a first non-specific identifier associated with said control indicator;
a second non-specific identifier associated with said positive indicator; and
a code for obtaining test result information from an external source based on said first and second indicators; and
a specimen collector.

9. A kit according to claim 8, further including a specimen developer.

10. A kit according to claim 9, said code identifying said testing apparatus from among a plurality of testing apparati.

11. A kit according to claim 10, said code identifying said testing apparatus by lot.

12. A kit according to claim 9, said code including intrinsic encoding key information for said first and second identifiers and said indicators.

13. A kit according to claim 9, wherein said control indicator is a positive control indicator.

14. A kit according to claim 9, further including a third indicator selected from the group consisting of a positive indicator, a positive control indicator, and a negative control indicator, the apparatus including a third non-specific identifier associated with said third indicator.

15. A kit according to claim 14, said testing apparatus further including a fourth indicator, said fourth indicator being selected from the group consisting of a positive indicator, a positive control indicator and a negative control indicator, the apparatus including a fourth non-specific identifier associated with said fourth indicator.

16. A method for manufacturing a testing apparatus, the method comprising the steps in any appropriate order of:
providing a sample tester, said sample testing including at least one control indicator and at least one positive indicator;
associating a first non-specific identifier with said control indicator and associating a second nonspecific identifier with said second indicator; and
applying a code to said sample tester.

17. A method for processing a plurality of incoming inquiries, each of said inquiries being initiated by one of a plurality of inquirers, each of said inquirers having been provided with a testing apparatus that comprises a specimen tester, said specimen tester including at least one control indicator and at least one positive indicator; a first non-specific identifier associated with said control indicator; a second nonspecific identifier associated with said positive indicator; and a code for obtaining test result information based on said first and second indicators, the method comprising the steps in any appropriate order of:
receiving said code from an inquirer;
receiving first indicator information associated with said first non-specific indicator;
receiving second indicator information associated with said second non-specific identifier;
determining test result information based on said code number, said first indicator information, and said second indicator information; and
providing said test result information to said inquirer.

18. A method according to claim 17, said inquiry being an anonymous inquiry received from one of a group of known users.

19. A method according to claim 17, said test being a medical test.

20. A method according to claim 19, said test having a positive result and a negative result, the method further comprising routing said inquiry based upon said test result.

21. A method according to claim 20, comprising the steps of routing said inquiry to an automated inquiry handler if said test result is negative and routing said inquiry to a live inquiry handler if said test result is positive.

22. A testing apparatus comprising:
a specimen tester, said specimen tester having a non-specific result display, said specimen tester including a code for obtaining test result information from an external source based on said non-specific result display.

23. A method for processing a plurality of incoming inquiries, each of said inquiries being initiated by one of a plurality of inquirers, each of said inquirers having been provided with a testing apparatus that comprises a specimen tester, said specimen tester having a non-specific result display and a code for obtaining test result information based on said nonspecific result display, the method comprising the steps in any appropriate order of:
receiving said code from an inquirer;
receiving result display information;
determining test result information based on said code number and said result display information; and
providing said test result information to said inquirer.

24. A method for obtaining test result information, comprising:
receiving a testing apparatus comprising:
a specimen tester, said specimen tester including at least one control indicator and at least one positive indicator;
a first non-specific identifier associated with said control indicator;
a second non-specific identifier associated with said positive indicator; and
a code for obtaining test result information from an external source based on said first and second indicators;
introducing a specimen to said specimen tester; contacting a result providing facility;
reporting result information from said first and second identifiers to said results providing facility; and
receiving test result information from said results reporting providing facility.

25. The method of claim 24, wherein said test result information is medical information.
